(19) United States
(12) Patent Application Publication
(10) Pub. No.: US 2022/0306994 A1
(43) Pub. Date: Sep. 29, 2022

(54) CHIMERIC CELLS COMPRISING DENDRITIC CELLS AND ENDOTHELIAL CELLS RESEMBLING TUMOR ENDOTHELIUM

(71) Applicant: Therapeutic Solutions International, Inc., Oceanside, CA (US)

(72) Inventors: Thomas E. ICHIM, Oceanside, CA (US); Timothy G. DIXON, Oceanside, CA (US); Feng LIN, Oceanside, CA (US); Famela RAMOS, Oceanside, CA (US); James VELTMEYER, Oceanside, CA (US)

(73) Assignee: Therapeutic Solutions International, Inc., Oceanside, CA (US)

(21) Appl. No.: 17/698,889

(22) Filed: Mar. 18, 2022

Related U.S. Application Data
(60) Provisional application No. 63/165,056, filed on Mar. 23, 2021.

(51) Int. Cl. C12N 5/16; C12N 15/86

(52) U.S. Cl. CPC C12N 5/16; C12N 15/86; C12N 2501/22; C12N 2501/26; C12N 2501/15; C12N 2501/165; C12N 2501/2304; C12N 2506/45; C12N 2740/15043

(57) ABSTRACT

Disclosed are means, methods and compositions of matter useful for induction of immunological responses towards tumor endothelial cells. In one embodiment the invention teaches fusion of dendritic cells and cells resembling tumor endothelial cells and administration of such chimeric cells as an immunotherapy for stimulation of tumor endothelial cell destruction. In other embodiments pluripotent stem cells are utilized to generate dendritic cells, wherein said dendritic cells are fused with pluripotent stem cell derived endothelial cells created in a manner to resemble tumor endothelial cells.

### CROSS-REFERENCE TO RELATED APPLICATIONS

This application claims priority to U.S. Provisional Application Ser. No. 63/165,056, filed on Mar. 23, 2021, entitled “Chimeric Cells Comprising Dendritic Cells and Endothelial Cells Resembling Tumor Endothelium”, which is incorporated herein by reference in its entirety.

### FIELD OF THE INVENTION

The field relates to compositions of matter useful for induction of immunological responses towards tumor endothelial cells, including the fusion of dendritic cells and cells resembling tumor endothelial cells.

### BACKGROUND OF THE INVENTION

The concept of treating cancer by blocking new blood vessel formation, angiogenesis, was pioneered by Judah Folkman who provided convincing arguments that it is not necessary to actively kill the tumor mass, but by suppressing its ability to grow through cutting off blood supply, malignant tumors may be converted into benign masses that eventually regress [1, 2]. Unfortunately, despite discovery of angiostatin, and endostatin, naturally derived inhibitors of angiogenesis, neither of these approaches translated into successful therapies. Nevertheless, the concept of targeting new blood vessel formation led to thousands of publications describing various antiangiogenic agents, of which several eventually proceeded through clinical trials and regulatory approval. Broadly anti-angiogenic agents approved by regulators can be classified into antibodies, such as Bevacizumab (Avastin) which binds VEGF [3], and Ramucirumab (Cyramza) [4], which binds VEGF-R2, as well as small molecules which bind multiple receptor kinases associated with angiogenesis such as Sunitinib [5-7], Cabozantinib [8-11], Pazopanib [12-14], and Regorafenib [15-17].

These approaches have augmented the standard of care for various tumor types and have achieved some level of progress. Unfortunately, the concept of blocking angiogenesis of cancer was not as simple as originally envisioned. One of the major hurdles in blocking angiogenesis was that even though de novo blood vessels are derived from nonmalignant cells, the malignant cells appear to possess ability to induce mutations in the new blood vessels. One example of the heterogeneity of tumor endothelial cells compared to endothelial cells from low and high metastatic tumors by Ohga et al [18]. The investigators extracted two types of tumor endothelial cells (TEM) from high-metastatic (HM) and low-metastatic (LM) tumors and compared their characteristics. HM tumor-derived TECs (HM-TECs) showed higher proliferative activity and invasive activity than LM tumor-derived TECs (LM-TECs). Moreover, the mRNA expression levels of pro-angiogenic genes, such as vascular endothelial growth factor (VEGF) receptors 1 and 2, VEGF, and hypoxia-inducible factor-1a, were higher in HM-TECs than in LM-TECs. The tumor blood vessels themselves and the surrounding area in HM tumors were exposed to hypoxia. Furthermore, HM-TECs showed higher mRNA expression levels of the stemness-related gene stem cell antigen and the mesenchymal marker CD90 compared with LM-TECs. HM-TECs were spheroid, with a smoother surface and higher circularity in the stem cell spheroid assay. HM-TECs differentiated into osteogenic cells, expressing activated alkaline phosphatase in an osteogenic medium at a higher rate than either LM-TECs or normal ECs. Furthermore, HM-TECs contained more aneuploid cells than LM-TECs. The investigators concluded that the results indicate that TECs from HM tumors have a more pro-angiogenic phenotype than those from LM tumors. It appears that the aggressiveness of the tumor not only can alter endothelial cell function but also drug resistance ability. In another study, Akiyama et al. [19]compared murine TECs and normal ECs. It was found that TECs were more resistant to paclitaxel with the up-regulation of multidrug resistance (MDR) 1 mRNA, which encodes the P-glycoprotein, compared with normal ECs. Normal human microvascular ECs were cultured in tumor-conditioned medium (CM) and became more resistant to paclitaxel through MDR1 mRNA up-regulation and nuclear translocation of Y-box-binding protein 1, which is an MDR1 transcription factor. Vascular endothelial growth factor (VEGF) receptor 2 (VEGFR2) and Akt were activated in human microvascular ECs by tumor CM. The investigators observed that tumor CM contained a significantly high level of VEGF. A VEGFR kinase inhibitor, Ki8751, and a phosphatidylinositol 3-kinase-Akt inhibitor, LY294002, blocked tumor CM-induced MDR1 up-regulation. MDR1 up-regulation, via the VEGF-VEGFR pathway in the tumor microenvironment, is one of the mechanisms of drug resistance acquired by TECs. It was observed that VEGF secreted from tumors up-regulated MDR1 through the activation of VEGFR2 and Akt. This process is a novel mechanism of the acquisition of drug resistance by TECs in the tumor microenvironment. Yet another study demonstrated that tumors can induce a “dedifferentiation” of tumor endothelium. Specifically, compared with NECs, stem cell markers such as Sca-1, CD90, and multidrug resistance 1 are upregulated in TECs, suggesting that stem-like cells exist in tumor blood vessels. TECs and NECs were isolated from melanoma-xenografted nude mice and normal dermis, respectively. The stem cell marker aldehyde dehydrogenase (ALDH) mRNA expression and activity were higher in TECs than those in NECs. Next, ALDHhigh/low TECs were isolated by fluorescence-activated cell sorting to compare their characteristics. Compared with ALDHlow TECs, ALDHhigh TECs formed more tubes on Matrigel-coated plates and sustained the tubular networks longer. Furthermore, VEGFR2 expression was higher in ALDHhigh TECs than that in ALDHlow TECs. In addition, ALDH was expressed in the tumor blood vessels of in vivo mouse models of melanoma and oral carcinoma, but not in normal blood vessels. These findings indicate that ALDHhigh TECs exhibit an angiogenic phenotype. Stem-like TECs may have an essential role in tumor angiogenesis [20].

What is it that causes the tumor to evoke changes in the endothelium? As suggested above, there is some support for growth factor mediated alterations, additionally, horizontal gene transfer may also play a role [21-29]. Although the field of horizontal gene transfer has historically been controversial one of the strongest evidences supporting this concept is the phenomena of donor-derived relapse in leukemic patients. In these situations patients with leukemia who relapse after bone marrow transplant have the relapsing cells originate from donor cells that transformed into malignant cells [30, 31]. Another issue that affected efficacy of anti-angiogenesis therapies is that in some tumors, the tumor cells themselves transdifferentiate into endothelial-like cells, termed tumor vascular channels, which possess ability to mutate around either antibody or kinase inhibitor drugs [32-37].

The previously mentioned means by which tumor endothelial cells can protect themselves against anti-angiogenic agents has resulted in relatively low clinical efficacy of these drugs. To understand the general lack of efficacy in the initial registration trialⁱⁱ, median progression free survival (PFS) of ovarian cancer patients who received bevacizumab plus chemotherapy was 6.8 months (95 percent CI: 5.6, 7.8) compared with 3.4 months (95 percent CI: 2.1, 3.8) for those who received chemotherapy alone. There was no statistically significant difference in overall survival (OS) for patients treated with bevacizumab plus chemotherapy compared with chemotherapy alone (median OS: 16.6 months versus 13.3 months; HR 0.89; 95 percent CI: 0.69, 1.14). Subset analysis led to identification that the group of patients that received paclitaxel with the antibody had the largest improvement, resulting in a 5.7-month improvement in median PFS (9.6 months versus 3.9 months; HR 0.47; 95 percent CI: 0.31, 0.72), an improvement in the objective response rate (53 percent versus 30 percent), and a 9.2-month improvement in median OS (22.4 months versus 13.2 months, HR 0.64; 95 percent CI: 0.41, 1.01)ⁱⁱⁱ. Multiple other trials where conducted for different indications using bevacizumab, unfortunately, progression free survival and overall survival was not increase more than a year in any of the studies [38-42], and neither in studies with small molecule kinase inhibitors [43-48].

This clinical translation, although highly beneficial in some patients, overall the effect was mediocre, highlights the disparity between animal studies, in which some studies complete regression was observed in established tumors [49, 50], whereas in clinical trials, relatively minimal effect compared to animal studies was observed [51]. One lesson from these studies is that the large heterogeneity of the patient and of the tumors, which calls for large patient populations in order to achieve an overall survival advantage.

Innovations in pharmacogenomics and personalized medicine will help identify specific patients and tumors that are likely to respond. Unfortunately, at present, patients with metastatic disease have limited options and a statistically significant extension of survival does equate to large market demand, as seen by the overall sale of angiogenesis inhibitors for cancer being over 20 billion annually.

### SUMMARY

Preferred embodiments are directed to a hybrid cell comprising of: a) a dendritic or dendritic like cell and b) an endothelial cell generated in a manner to resemble tumor endothelium.

Preferred hybrid cells include embodiments wherein said hybrid cells is generated by fusion of a dendritic or dendritic like cell and an endothelial cell generated in a manner to resemble tumor endothelium.

Preferred hybrid cells include embodiments wherein said fusion is created by placement of both cell types in physical proximity while treating both cells with an agent capable of causing fusion of plasma membrane.

Preferred hybrid cells include embodiments wherein said fusion agent is polyethylene glycol.

Preferred hybrid cells include embodiments wherein said fusion agent is ultrasound waves.

Preferred hybrid cells include embodiments wherein said fusion agent is radio waves.

Preferred hybrid cells include embodiments wherein said fusion agent is phosphatidylcholine.

Preferred hybrid cells include embodiments wherein said dendritic cell is generated from a stem cell.

Preferred hybrid cells include embodiments wherein said stem cell is a pluripotent stem cell.

Preferred hybrid cells include embodiments wherein said pluripotent stem cell is an inducible pluripotent stem cell.

Preferred hybrid cells include embodiments wherein said pluripotent stem cell is a parthenogenic stem cell.

Preferred hybrid cells include embodiments wherein said pluripotent stem cell is a somatic cell nuclear transfer derived stem cell.

Preferred hybrid cells include embodiments wherein said pluripotent stem cell is generated by cytoplasmic transfer from an immature cell to a mature cell.

Preferred hybrid cells include embodiments wherein said dendritic cell is generated by collection of embryoid bodies from said pluripotent stem cells.

Preferred hybrid cells include embodiments wherein said embryoid bodies are dissociated and cells are cultured in cytokines capable of expanding dendritic cell progenitors.

Preferred hybrid cells include embodiments wherein said dendritic cell progenitors are cultured in GM-CSF.

Preferred hybrid cells include embodiments wherein said dendritic cell progenitors are cultured in flt-3 ligand.

Preferred hybrid cells include embodiments wherein said dendritic cell progenitors are cultured in IL-4.

Preferred hybrid cells include embodiments wherein said endothelial cells are derived from a pluripotent stem cell.

Preferred hybrid cells include embodiments wherein said pluripotent stem cell is an inducible pluripotent stem cell.

Preferred hybrid cells include embodiments wherein said pluripotent stem cell is a parthenogenic stem cell.

Preferred hybrid cells include embodiments wherein said pluripotent stem cell is a somatic cell nuclear transfer derived stem cell.

Preferred hybrid cells include embodiments wherein said pluripotent stem cell is generated by cytoplasmic transfer from an immature cell to a mature cell.

Preferred hybrid cells include embodiments wherein said hybrid cell is utilized to induce an immune response against tumor endothelial cells.

Preferred hybrid cells include embodiments wherein said endothelial cells are generated by culture of endothelial progenitor cells in a media replicating the tumor microenvironment.

Preferred hybrid cells include embodiments wherein said endothelial cells are generated by culture of endothelial progenitor cells in a media replicating the tumor microenvironment.

Preferred hybrid cells include embodiments wherein said media contains prostaglandin E2.

Preferred hybrid cells include embodiments wherein said media contains TGF-beta.

Preferred hybrid cells include embodiments wherein said media contains IL-10.

Preferred hybrid cells include embodiments wherein said media contains VEGF.

Preferred hybrid cells include embodiments wherein said media contains PDGF-BB.

Preferred hybrid cells include embodiments wherein said media contains EGF.

Preferred hybrid cells include embodiments wherein said media contains FGF-1.

Preferred hybrid cells include embodiments wherein said media contains FGF-2.

### BRIEF DESCRIPTION OF THE DRAWINGS

FIG. 1 is a bar graph showing tumor volume based on cell type.

### DETAILED DESCRIPTION OF THE INVENTION

The invention provides a chimeric cell capable of stimulating immunity to tumor endothelial cells by fusing dendritic cells derived from iPSC together with in vitro generated endothelial cells which resemble the tumor endothelium.

“Marker” and “Biomarker” are used interchangeably to refer to a gene expression product that is differentially present in a samples taken from two different subjects, e.g., from a test subject or patient having (a risk of developing) an ischemic event, compared to a comparable sample taken from a control subject (e.g., a subject not having (a risk of developing) an ischemic event; a normal or healthy subject). Alternatively, the terms refer to a gene expression product that is differentially present in a population of cells relative to another population of cells.

The phrase “differentially present” refers to differences in the quantity or frequency (incidence of occurrence) of a marker present in a sample taken from a test subject as compared to a control subject. For example, a marker can be a gene expression product that is present at an elevated level or at a decreased level in blood samples of a risk subjects compared to samples from control subjects. Alternatively, a marker can be a gene expression product that is detected at a higher frequency or at a lower frequency in samples of blood from risk subjects compared to samples from control subjects.

A gene expression product is “differentially present” between two samples if the amount of the gene expression product in one sample is statistically significantly different from the amount of the gene expression product in the other sample. For example, a gene expression product is differentially present between two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

As used herein, the terms “antibody” and “antibodies” refer to monoclonal antibodies, multispecific antibodies, synthetic antibodies, human antibodies, humanized antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies of the present invention include immunoglobulin molecules and immunologically active portions of immunoglobulin molecules, i.e., molecules that contain an antigen binding site that immunospecifically binds to a polypeptide antigen encoded by a gene comprised in the genomic regions or affected by genetic transformations in the genomic regions listed in Table 1. The immunoglobulin molecules of the invention can be of any type (e.g., IgG, IgE, IgM, IgD, IgA and IgY), class (e.g., IgG.sub.1, IgG.sub.2, IgG.sub.3, IgG.sub.4, IgA.sub.1 and IgA.sub.2) or subclass of immunoglobulin molecule.

“Immunoassay” is an assay that uses an antibody to specifically bind an antigen (e.g., a marker). The immunoassay is characterized by the use of specific binding properties of a particular antibody to isolate, target, and/or quantify the antigen. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

The phrase “specifically (or selectively) binds” when referring to an antibody, or “specifically (or selectively) immunoreactive with”, when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein.

The terms “affecting the expression” and “modulating the expression” of a protein or gene, as used herein, should be understood as regulating, controlling, blocking, inhibiting, stimulating, enhancing, activating, mimicking, bypassing, correcting, removing, and/or substituting said expression, in more general terms, intervening in said expression, for instance by affecting the expression of a gene encoding that protein.

“Allogeneic,” as used herein, refers to cells of the same species that differ genetically from cells of a host.

“Autologous,” as used herein, refers to cells derived from the same subject. The term “engraft” as used herein refers to the process of stem cell incorporation into a tissue of interest in vivo through contact with existing cells of the tissue.

“Approximately” or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the current disclosure, and vice versa. Furthermore, compositions of the current disclosure can be used to achieve methods of the current disclosure.

“Somatic cell” it is meant any cell in an organism that has differentiated sufficiently, so that in the absence of experimental manipulation, does not ordinarily give rise to cells of all three germ layers of the body, i.e., ectoderm, mesoderm and endoderm. “Somatic cell” includes “multipotent cells” (i.e., progenitor cells), but does not include “pluripotent” or “totipotent cells.” For example, somatic cells would include both neurons and neural progenitors, the latter of which may be able to naturally give rise to all or some cell types of the central nervous system but cannot give rise to cells of the mesoderm or endoderm lineages.

“Multipotency” is referred to herein in the context of multipotent progenitor cells which have the potential to give rise to multiple cell types, but are less potent (more limited in their differentiation potential) than a pluripotent stem cell. For example, a multipotent stem cell is a hematopoietic cell that can develop into several types of blood cells, but cannot develop into brain cells or other types of cells.

“Pluripotent” is referred to herein as the property of a cell/cell type as having the potential to differentiate into any of the three germ layers: endoderm (e.g., interior stomach lining, gastrointestinal tract, the lungs), mesoderm (e.g., muscle, bone, blood, urogenital), or ectoderm (e.g., epidermal tissues and nervous system).

“Pluripotent stem cells” include natural pluripotent stem cells and induced pluripotent stem cells. They can give rise to any fetal or adult cell type. However, alone they generally cannot develop into a fetal or adult organism because they lack the potential to contribute to extra-embryonic tissue, such as the placenta.

“Induced pluripotent stem cells” or (“iPSCs”) are similar to natural pluripotent stem cells, such as embryonic stem cells, in many aspects, such as the expression of certain stem cell genes and/or proteins, chromatin methylation patterns, doubling time, embryoid body formation, teratoma formation, viable chimera formation, and potency and differentiability. Induced pluripotent cells may be derived from for example, adult stomach, liver, skin cells and blood cells (e.g., cord blood cells). iPSCs may be derived by transfection of synthetic transcription factors and/or certain stem cell-associated genes into non-pluripotent (e.g., somatic) cells. In certain embodiments, transfection may be achieved through viral vectors, such as retroviruses, for example, and non-viral or episomal vectors. Transfected genes can include, but are not limited to, reprogramming factors Oct3/4 (Pou5f1), Klf-4, c-Myc, Sox-2, Nanog and Lin28. Sub-populations of transfected cells may begin to become morphologically and biochemically similar to pluripotent stem cells, and can be isolated through morphological selection, doubling time, or through a reporter gene and antibiotic selection.

“Peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

As used herein, “cancer” refers to all types of cancer or neoplasm or malignant tumors found in animals, including leukemias, carcinomas and sarcomas. Examples of cancers are cancer of the brain, melanoma, bladder, breast, cervix, colon, head and neck, kidney, lung, non-small cell lung, mesothelioma, ovary, prostate, sarcoma, stomach, uterus and Medulloblastoma. The term “leukemia” is meant broadly progressive, malignant diseases of the hematopoietic organs/systems and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemi.

The term “carcinoma” refers to a malignant new growth made up of epithelial cells tending to infiltrate the surrounding tissues, and/or resist physiological and non-physiological cell death signals and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrmcous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti, The term “sarcoma” generally refers to a tumor which is made up of a substance like the embryonic connective tissue and is generally composed of closely packed cells embedded in a fibrillar, heterogeneous, or homogeneous substance. Sarcomas include, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma. Additional exemplary neoplasias include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

In some particular embodiments of the disclosure, the cancer treated is a melanoma. The term “melanoma” is taken to mean a tumor arising from the melanocytic system of the skin and other organs. Melanomas include, for example, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma. In one embodiment, stressed cells are recognized by the modified immune cells of the disclosure. Stressed cells may be recognized by immune cells that selectively are activated in response to proteins associated with stress such as heat shock proteins. Conditions that the disclosure may be useful for diagnosing are selected from the group consisting of: motor-neuron disease, multiple sclerosis, degenerative diseases of the CNS, dementia, Alzheimer's Disease, Parkinson's Disease, cerebrovascular accidents, epilepsy, temporary ischaemic accidents, mood disorders, psychotic illness, specific lobe dysfunction, pressure related CNS injury, cognitive dysfunction, deafness, blindness, anosmia, motor deficits, sensory deficits, head injury, trauma to the CNS, arrhythmias, myocardial infarction, pericarditis, congestive heart disease, valve related pathologies, myocardial dysfunction, endocardial dysfunction, pericardial dysfunction, sclerosis and thickening of valve flaps, fibrosis of cardiac muscle, decline in cardiac reserve, congenital defects of the heart or circulatory system, developmental defects of the heart or circulatory system, hypoxic or necrotic damage, blood vessel damage, cardiovascular disease (for example, angina, dissected aorta, thrombotic damage, aneurysm, atherosclerosis, emboli damage), disorders of the sweat gland, disorders of the sebaceous gland, piloerectile dysfunction, follicular problems, hair loss, epidermal disease, disease of the dermis or hypodermis, burns, ulcers, sores, infections, striae, seborrhoea, rosacea, disorders of the musculoskeletal system including disease and damage to muscles and bones, endocondral ossification, osteoporosis, osteomalacia, rickets, pagets disease, rheumatism, arthritis, diseases of the endocrine system, diseases of the lymphatic system, diseases of the urinary system, diseases of the reproductive system, metabolic diseases, diseases of the sinus, diseases of the nasopharynx, diseases of the oropharynx, diseases of the laryngopharynx, diseases of the larynx, diseases of the ligaments, diseases of the vocal cords, vestibular folds, glottis, epiglottis, trachea, mucocilliary mucosa, trachealis muscles, emphysema, chronic bronchitis, pulmonary infection, asthma, tuberculosis, cystic fibrosis, diseases of gas exchange, burns, barotraumas, dental care, periodontal disease, deglutination problems, ulcers, enzymatic disturbances/deficiencies, fertility problems, paralysis, dysfunction of absorption or absorptive services, diverticulosis, inflammatory bowel disease, hepatitis, cirrhosis, portal hypertension, diseases of sight, and cancer.

As used herein, “immune cell” refers to a cell capable of interacting with a cell that has abnormal qualities. Immune cells may include cells classically known to play a role in the immune system, such as T cells, B cells, and NK cells, or cells that are not classically considered immune cells but play a role in the identification of pathology. The cells include mesenchymal stem cells, hematopoietic stem cells or progeny thereof, and monocytes. In some embodiments immune cells are autologous to the recipient, or in other embodiments immune cells are allogeneic. In some specific embodiments, allogeneic cells are used that possess reduced allogenicity. Immune cells can include, for example, B cells, T cells, innate lymphoid cells, natural killer cells, natural killer T cells, gamma delta T cells, T regulatory cells, macrophages, monocytes, dendritic cells, neutrophils, myeloid derived suppressor cells.

“Redirected immune cell” refers to an immune cell, which has been modified to specifically recognize characteristics associated with an abnormal cell. In one specific example a “redirected immune cell” refers to a CAR-T cell, in another specific embodiment a “redirected immune cell refers to a cell made to express a non-endogenous receptor, wherein the non-endogenous receptor allows for a specific interaction with an abnormal cell.

As used herein, the term “population of immune cells” refers to one or more immune cells, such as a group of immune cells.

“Obtaining a population of immune cells” can be achieved by removal of a sample from a subject and purifying the population of immune cells. The population of immune cells may be obtained, for example, by obtaining a sample having a population of immune cells, including a blood sample, a tissue sample, or a biological fluid sample. The sample may be obtained by withdrawing blood or biological fluid from a subject or by removal of cells, tumors, or tissues from a subject.

“Delivery vehicle” refers to a molecule or composition useful for holding or suspending and transporting a compound in vivo for the purpose of localization and detection or release and delivery of the transported compound. Delivery vehicles can include, for example, B cells, T cells, innate lymphoid cells, natural killer cells, natural killer T cells, gamma delta T cells, T regulatory cells, macrophages, monocytes, dendritic cells, neutrophils, myeloid derived suppressor cells, mast cells, hematopoietic stem cells, fibroblasts, stromal vascular fraction, exosomes, endothelial progenitor cells, mesenchymal stem cells, pluripotent cell lines, or engineered nanoparticles. The delivery vehicle may be obtained by manufacture or by removal of a sample from a subject and purifying a delivery vehicle from the sample.

“Binding” or “interaction” as used herein (e.g. with reference to a synthetic transcriptional modulator binding the polypeptide-binding segment of a guide RNA) refers to a non-covalent interaction between macromolecules (e.g., between DNA and RNA, or between a polypeptide and a polynucleotide). “Binding” may also be referred to as “associated with” or “interacting”. “Binding” as used herein means that the binding partners are capable of binding to each other (e.g., will not necessarily bind to each other). Some portions of a binding interaction may be sequence-specific, but not all components of a binding interaction need be sequence-specific (e.g., contacts with phosphate residues in a DNA backbone). Binding interactions are generally characterized by a dissociation constant (Kd), e.g., less than 1 mM, less than 100 uM, less than 10 uM, less than 1 uM, less than 100 nM, less than 10 nM. “Affinity” refers to the strength of binding, increased binding affinity being correlated with a lower Kd.

“Promoter,” “promoter sequence,” or promoter region” refers to a DNA regulatory region/sequence capable of binding RNA polymerase and involved in initiating transcription of a downstream coding or non-coding sequence. In some examples of the present disclosure, the promoter sequence includes the transcription initiation site and extends upstream to include the minimum number of bases or elements necessary to initiate transcription at levels detectable above background. In some embodiments, the promoter sequence includes a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the various vectors of the present invention.

“Vector” or “expression vector” is a replicon, such as a plasmid, phage, virus, or cosmid, to which another DNA segment, i.e. an “insert”, may be attached so as to bring about the replication of the attached DNA segment in a cell. “Vector” includes episomal (e.g., plasmids) and non episomal vectors. In some embodiments of the present disclosure the vector is an episomal vector, which is removed/lost from a population of cells after a number of cellular generations, e.g., by asymmetric partitioning.

The invention provides means of utilizing the potent antigen presenting activities of the dendritic cell together with tumor endothelial markers generated from in vitro iPSC derived endothelial cells cultured under conditions that resemble the tumor microenvironment. The angiogenic process is a very dynamic and fluid process. Although VEGF is critical in formation of new blood vessels, there are multiple other cytokines that possess redundant roles. Many times resistance to VEGF signaling is a result of tumor endothelial cells becoming more reliant on other cytokines that take over the function of VEGF pathway [52, 53]. Additionally, mutations in VEGF-R2 have been identified in tumor endothelium, which has been associated with non-responsiveness [54]. Dose escalation of VEGF inhibiting antibodies is limited by different toxicities such as cardiac toxicities [55-61]. An interesting observation is that although hematopoietic stem cells are known to utilize VEGF-R2 for self-renewal, cytopenias are generally not observed in patients receiving VEGF pathway blockers [62, 63]. Kinase inhibitors also suffer from mutations of active sites, as well as off target toxicities. For example, a study in colon patients receiving sunitinib demonstrated mutations in all major kinases associated with endothelial proliferation [64].

Thus limitations of efficacy of anti-angiogenesis approaches that are currently in the clinic appear to be associated with: a) targeting of only one pathway allows the tumor endothelium to start utilizing other pathways; and b) small molecule inhibitors are slow in onset of action, which allows for time to pass and mutations to accumulate.

Immunological targeting of angiogenesis may be a more promising approach due to: a) ability of immune system to “mutate” with cancer endothelium, thus overcoming ability of molecular evasion; b) more rapid onset of immune attack, including direct killing of endothelium may not allow enough time for tumor endothelium to mutate and/or acquire resistant properties.

Classical studies have shown that tumor infiltrating lymphocytes correlate with positive prognosis in various tumors [65-77]. The invention teaches means of increasing lymphocytic recognition of cancer endothelial cells. Unfortunately, there are several important factors that prevent efficacy of infiltrating lymphocytes. Firstly, tumor masses originate from tumor stem cells, which possess distinctly different antigenic composition [78-82]. Accordingly, infiltration of lymphocytes, while useful for targeting tumor stem cell progeny, may not actually reach, or recognize tumor stem cells. This is also relevant in light of studies showing tumor stem cells possess various immune evasive molecules such as DAF, IL-10 and HLA-G. Secondly, tumors are known to possess high interstitial pressure, which physically limits ability of lymphocytes to enter the tumor mass, which often possesses necrotic tissue. Thirdly, tumor acidosis, hypoxia, and high adenosine concentrations have been demonstrated to selectively inhibit cytotoxic cells and promote T regulatory cells.

Despite theoretical obstacles to efficacy of immunotherapy of tumors, numerous studies have shown that in some patients, cancer vaccines, ranging from whole cell lysates of the 1970s to defined peptide vaccines, to nucleic acid vaccines, all induce in select patients some level of tumor regression. While in double blind trials these approaches often fail, due to heterogeneity of patients and tumors, in some patients documented durable responses are observed. It is not the scope of this paper to speculate on the heterogeneity, however, factors, which were not appreciated in previous studies, including polymorphisms in immune associated genes, expression of different mutations, diet, and gut microbiota content may all contribute observations of tumor eradication in some patients whereas in other patients no effect or even acceleration of tumor. Regardless of cause, it is documented that some patients highly respond to active vaccination against tumor antigens.

We propose that significantly higher killing of tumor cells may be achieved by directing antigen-specific immune responses toward the tumor endothelium. In contrast, to tumor stem cells, which are the desired target of an effective vaccination program, tumor endothelial cells are directly in contact with the circulatory system, thus permitting uninhibited access to immune cells and antibodies. Additionally, tumor endothelium is known to possess an increased level of prothrombotic molecules such as tissue factor [83-87]. Thus hypothetically, stimulation of thrombosis may be induced at a reduced threshold by immune cells/molecules targeting tumor angiogenesis as compared to existing vasculature in the body, which possesses numerous antithrombotic activities.

Early studies demonstrated that xenogeneic immunization with angiogenic proteins resulted in tumor regression. In some embodiments of the invention iPSC derived endothelial cells are utilized in a xenogeneic manner. Breaking of self-tolerance using xenogeneic proteins is commonly used to elicit autoimmunity in models such as collagen induced arthritis and experimental autoimmune encephalitis (EAE). Accordingly, previous studies have shown that while inhibition of tolerance to self-proteins associated with oncogenic angiogenesis results in inhibition of tumor growth, alterations to physiological processes such as wound healing or menstruation where not observed. This perhaps indicates the ability of the immune system to differentiate between pathological conditions of angiogenesis versus physiological. One potential analogy are clinical studies using antigen specific T cells for autoimmunity as a vaccine. In these studies, despite T cells being administered in an immunogenic manner, only antigen specific, idiotypic T cells are immunologically attacked and not all T cells.

A fundamental question determining feasibility of vaccine-induced killing of tumor vasculature is whether antigens exist on the tumor endothelium that are not expressed on physiologically normal blood vessels, and whether immunity could be raised against such antigens. The roundabout receptor (ROBO)-4 is a transmembrane protein that was originally found to orchestrate the neuronal guidance mechanism of the nervous system [88]. ROBO4 was found to be selectively expressed on tumor endothelial cells but not healthy vasculature [89]. Zhuang et al demonstrated that mice immunized with the extracellular domain of mouse Robo4, showed a strong antibody response to Robo4, with no objectively detectable adverse effects on health, including normal menstruation and wound healing. Robo4 vaccinated mice showed impaired fibrovascular invasion and angiogenesis in a rodent sponge implantation assay, as well as a reduced growth of implanted syngeneic Lewis lung carcinoma. The anti-tumor effect of Robo4 vaccination was present in CD8 deficient mice but absent in B cell or IgG1 knockout mice, suggesting antibody dependent cell mediated cytotoxicity (ADCC) as the anti-vascular/anti-tumor mechanism [90]. Another antigen that is more ubiquitously found throughout the body, but with higher expression on tumor endothelial cells is the VEGF receptor 2 (VEGFR2) which is typically found on hematopoietic stem cells and endothelial progenitor cells [62, 63, 91-94]. Despite expression on non-malignant tissue, successful induction of antitumor immunity has been demonstrated using various immunization means against this antigen. Yan et al utilized irradiated AdVEGFR2-infected cell vaccine-based immunotherapy in the weakly immunogenic and highly metastatic 4T1 murine mammary cancer model. Lethally irradiated, virus-infected 4T1 cells were used as vaccines. Vaccination with lethally irradiated AdVEGFR2-infected 4T1 cells inhibited subsequent tumor growth and pulmonary metastasis compared with challenge inoculations. Angiogenesis was inhibited, and the number of CD8+ T lymphocytes was increased within the tumors. Antitumor activity was also caused by the adoptive transfer of isolated spleen lymphocytes, thus demonstrating induction of tumor specific immunity [95]. Other approaches have been utilized to induce immunity to VEGFR2, which resulted in induction of tumor regression without systemic toxicities [96-101]. Other approaches have been utilized to induce immunity to VEGFR2, which resulted in induction of tumor regression without systemic toxicities [96-101]. Tumor endothelial marker 1 or endosialin is another antigen found selectively on the tumor vasculature. Facciponte et al demonstrated that a DNA vaccination targeting endosialin reduced tumor vascularity, increased CD3+ T cell infiltration, and was correlated with significant inhibition of tumor growth. Epitope spreading to tumor antigens following the initial immune response against the tumor vasculature gives evidence that targeting the tumor endothelium may activate a cascade of pathways conducive to tumor regression. Additionally, the DNA vaccination against endosialin did not affect other angiogenesis dependent physiological processes, exhibiting no adverse effects on menstruation, embryonic development, pregnancy, and wound healing in mouse models [102]. Other markers associated with tumor blood vessels have been utilized therapeutically in animal models for vaccination purposes including survivin [103-105], endosialin [106], and xenogeneic FGF2R [107], VEGF [108], VEGF-R2 [109], MMP-2 [110], and endoglin [111, 112].

At a clinical level, previous studies have shown lack of autoantibody responses and signals of efficacy subsequent to immunization of patients using human umbilical vein endothelial cells (HUVEC). In our studies, ValloVax is prepared from placental endothelial cells, which are induced to proliferate and treated with interferon gamma to augment immunogenicity. We have previously reported lack of autoantibody response, clinical safety, and specific antibody elicitation toward tumor endothelial antigens.

### EXAMPLE

iPSCs (ATCC-DYR0100 Human Induced Pluripotent Stem (IPS) Cells (ATCC® ACS-1011™) were cultured on feeder layers of OP9 cells for 6 to 7 days in α-MEM supplemented with 20% FBS. The mesodermally differentiated cells were then harvested, reseeded onto fresh OP9 cell layers, and cultured in α-MEM supplemented with 20% FBS, 20 ng/mL GM-CSF, and 50 μmol/L 2-ME. On day 13 to 14, floating cells were recovered by pipetting. These cells were considered to be iPSC-derived myeloid cells (iPS-MCs). The cells were infected with lentivirus vectors expressing the c-Myc and the Brother of the Regulatory of Imprinted Sites (BORIS) gene, as well as shRNA encoding siRNA silencing VEGF-R in the presence of 8 ng/mL polybrene (Sigma-Aldrich), and were cultured in α-MEM supplemented with 20% FBS, 30 ng/mL GM-CSF, and 30 ng/mL M-CSF. After 5 to 6 days, proliferating cells appeared and were considered to be ESC- or iPSC-derived pMCs (ES-pMC or iPS-pMC, respectively). To induce the differentiation of these cells into DC-like cells (pMC-DC), they were cultured in RPMI-1640 supplemented with 20% FBS in the presence of 20 ng/mL IL4 plus 30 ng/mL GM-CSF for 3 days. These cells where fused with endothelial cells derived from iPSC cultured under conditions replicating tumor microenvironment, specifically, cells were grown in 10 ng/ml PGE2, 100 pg/ml TGF-beta, and 100 pg/ml VEGF. Cells were cultured for 7 days and subsequently sorted for expression of the cancer endothelial marker TEM-1. Fusion between the two cells was performed using the polyethelyne glycol method utilized to generate monoclonal antibodies.

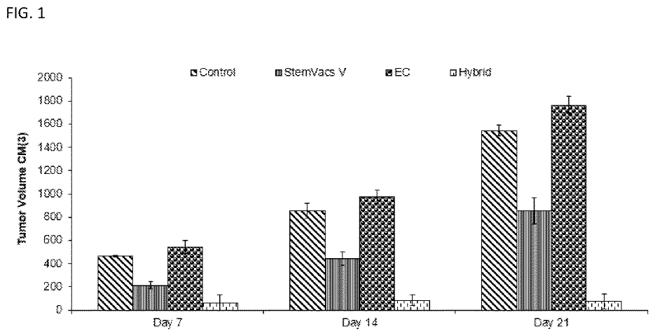

Mice were inoculated with 500,000 lewis lung carcinoma cells. Mice were injected with saline (Control), BORIS expressing VEGF R silenced dendritic cells (StemVacs-V), iPSC derived endothelial cells (EC) and the hybrid (hybrid). Tumor growth was assessed by calipers. Results are shown in FIG. 1.

### REFERENCES

1. Cao, Y., et al., Forty-year journey of angiogenesis translational research. Sci Transl Med, 2011. 3(114): p. 114rv3.
2. Abdollahi, A. and J. Folkman, Evading tumor evasion: current concepts and perspectives of anti-angiogenic cancer therapy. Drug Resist Updat, 2010. 13(1-2): p. 16-28.
3. Chen, H. X., R. E. Gore-Langton, and B. D. Cheson, Clinical trials referral resource: Current clinical trials of the anti-VEGF monoclonal antibody bevacizumab. Oncology (Williston Park), 2001. 15(8): p. 1017, 1020, 1023-6.
4. Krupitskaya, Y. and H. A. Wakelee, Ramucirumab, a fully human mAb to the transmembrane signaling tyrosine kinase VEGFR-2 for the potential treatment of cancer. Curr Opin Investig Drugs, 2009. 10(6): p. 597-605.
5. Abrams, T. J., et al., SU11248 inhibits KIT and platelet-derived growth factor receptor beta in preclinical models of human small cell lung cancer. Mol Cancer Ther, 2003. 2(5): p. 471-8.
6. Carlisle, B., et al., Benefit, Risk, and Outcomes in Drug Development. A Systematic Review of Sunitinib. J Natl Cancer Inst, 2016. 108(1).
7. Izzedine, H., et al., Sunitinib malate. Cancer Chemother Pharmacol, 2007. 60(3): p. 357-64.
8. Viola, D., V. Cappagli, and R. Elisei, Cabozantinib (XL184) for the treatment of locally advanced or metastatic progressive medullary thyroid cancer. Future Oncol, 2013. 9(8): p. 1083-92.
9. Roy, S., et al., A novel multiple tyrosine-kinase targeted agent to explore the future perspectives of anti-angiogenic therapy for the treatment of multiple solid tumors: cabozantinib. Anticancer Agents Med Chem, 2015. 15(1): p. 37-47.
10. Tannir, N. M., G. Schwab, and V. Grunwald, Cabozantinib: an Active Novel Multikinase Inhibitor in Renal Cell Carcinoma. Curr Oncol Rep, 2017. 19(2): p. 14.
11. Yakes, F. M., et al., Cabozantinib (XL184), a novel MET and VEGFR2 inhibitor, simultaneously suppresses metastasis, angiogenesis, and tumor growth. Mol Cancer Ther, 2011. 10(12): p. 2298-308.
12. Gril, B., et al., The B-Raf status of tumor cells may be a significant determinant of both antitumor and anti-angiogenic effects of pazopanib in xenograft tumor models. PLoS One, 2011. 6(10): p. e25625.
13. van Geel, R. M., J. H. Beijnen, and J. H. Schellens, Concise drug review: pazopanib and axitinib. Oncologist, 2012. 17(8): p. 1081-9.
14. Ferrero, S., et al., Pharmacokinetic drug evaluation of pazopanib for the treatment of uterine leiomyosarcomas. Expert Opin Drug Metab Toxicol, 2017. 13(8): p. 881-889.
15. Gaumann, A. K., et al., Receptor tyrosine kinase inhibitors: Are they real tumor killers? Int J Cancer, 2016. 138(3): p. 540-54.
16. Miura, K., et al., The preclinical development of regorafenib for the treatment of colorectal cancer. Expert Opin Drug Discov, 2014. 9(9): p. 1087-101.
17. Rimassa, L., et al., Regorafenib for the treatment of unresectable hepatocellular carcinoma. Expert Rev Anticancer Ther, 2017. 17(7): p. 567-576.
18. Ohga, N., et al., Heterogeneity of tumor endothelial cells: comparison between tumor endothelial cells isolated from high-and low-metastatic tumors. Am J Pathol, 2012. 180(3): p. 1294-307.
19. Akiyama, K., et al., Tumor endothelial cells acquire drug resistance by MDR1 up-regulation via VEGF signaling in tumor microenvironment. Am J Pathol, 2012. 180(3): p. 1283-93.
20. Ohmura-Kakutani, H., et al., Identification of tumor endothelial cells with high aldehyde dehydrogenase activity and a highly angiogenic phenotype. PLoS One, 2014. 9(12): p. e113910.
21. Sruthi, T. V., et al., Horizontal transfer of miR-23a from hypoxic tumor cell colonies can induce angiogenesis. J Cell Physiol, 2017.
22. Kosaka, N., et al., Neutral sphingomyelinase 2 (nSMase2)-dependent exosomal transfer of angiogenic microRNAs regulate cancer cell metastasis. J Biol Chem, 2013. 288(15): p. 10849-59.
23. Zhang, Y., et al., Tumacrophage: macrophages transformed into tumor stem-like cells by virulent genetic material from tumor cells. Oncotarget, 2017. 8(47): p. 82326-82343.
24. Abdouh, M., et al., Exosomes isolated from cancer patients' sera transfer malignant traits and confer the same phenotype of primary tumors to oncosuppressor-mutated cells. J Exp Clin Cancer Res, 2017. 36(1): p. 113.
25. Semina, S. E., et al., Horizontal Transfer of Tamoxifen Resistance in MCF-7 Cell Derivates: Proteome Study. Cancer Invest, 2017. 35(8): p. 506-518.
26. Cal, J., et al., Functional transferred DNA within extracellular vesicles. Exp Cell Res, 2016. 349(1): p. 179-183.
27. Hamam, D., et al., Transfer of malignant trait to BRCA1 deficient human fibroblasts following exposure to serum of cancer patients. J Exp Clin Cancer Res, 2016. 35: p. 80.
28. Berridge, M. V., L. Dong, and J. Neuzil, Mitochondrial DNA in Tumor Initiation, Progression, and Metastasis: Role of Horizontal mtDNA Transfer. Cancer Res, 2015. 75(16): p. 3203-8.
29. Chen, W. X., et al., Exosomes from drug-resistant breast cancer cells transmit chemoresistance by a horizontal transfer of microRNAs. PLoS One, 2014. 9(4): p. e95240.
30. Zhu, X., et al., BCR-ABL1-positive microvesicles transform normal hematopoietic transplants through genomic instability: implications for donor cell leukemia. Leukemia, 2014. 28(8): p. 1666-75.
31. Stein, J., et al., Origin of leukemic relapse after bone marrow transplantation: comparison of cytogenetic and molecular analyses. Blood, 1989. 73(7): p. 2033-40.
32. Angara, K., T. F. Bonin, and A. S. Arbab, Vascular Mimicry: A Novel Neovascularization Mechanism Driving Anti-Angiogenic Therapy (AAT) Resistance in Glioblastoma. Transl Oncol, 2017. 10(4): p. 650-660.
33. Sood, A. K., et al., The clinical significance of tumor cell-lined vasculature in ovarian carcinoma: implications for anti-vasculogenic therapy. Cancer Biol Ther, 2002. 1(6): p. 661-4.
34. Mahase, S., et al., Hypoxia-Mediated Mechanisms Associated with Antiangiogenic Treatment Resistance in Glioblastomas. Am J Pathol, 2017. 187(5): p. 940-953.
35. Pinto, M. P., et al., Escaping Antiangiogenic Therapy: Strategies Employed by Cancer Cells. Int J Mol Sci, 2016. 17(9).
36. Schnegg, C. I., et al., Induction of Vasculogenic Mimicry Overrides VEGF-A Silencing and Enriches Stem-like Cancer Cells in Melanoma. Cancer Res, 2015. 75(8): p. 1682-90.
37. Soda, Y., et al., Transdifferentiation of glioblastoma cells into vascular endothelial cells. Proc Natl Acad Sci USA, 2011. 108(11): p. 4274-80.
38. Li, Q., et al., Angiogenesis inhibitors for the treatment of small cell lung cancer (SCLC): A meta-analysis of 7 randomized controlled trials. Medicine (Baltimore), 2017. 96(13): p. e6412.
39. Lombardi, G., et al., Effectiveness of antiangiogenic drugs in glioblastoma patients: A systematic review and meta-analysis of randomized clinical trials. Crit Rev Oncol Hematol, 2017. 111: p. 94-102.
40. Roviello, G., et al., The role of bevacizumab in solid tumours: A literature based meta-analysis of randomised trials. Eur J Cancer, 2017. 75: p. 245-258.
41. Shih, T. and C. Lindley, Bevacizumab: an angiogenesis inhibitor for the treatment of solid malignancies. Clin Ther, 2006. 28(11): p. 1779-802.
42. Chase, J. L., Clinical use of anti-vascular endothelial growth factor monoclonal antibodies in metastatic colorectal cancer. Pharmacotherapy, 2008. 28(11 Pt 2): p. 23S-30S.
43. Yu, J., et al., Efficacy and safety of angiogenesis inhibitors in advanced gastric cancer: a systematic review and meta-analysis. J Hematol Oncol, 2016. 9(1): p. 111.
44. Ciliberto, D., et al., A systematic review and meta-analysis of randomized trials on the role of targeted therapy in the management of advanced gastric cancer: Evidence does not translate? Cancer Biol Ther, 2015. 16(8): p. 1148-59.
45. Welsh, S. J. and K. Fife, Pazopanib for the treatment of renal cell carcinoma. Future Oncol, 2015. 11(8): p. 1169-79.
46. Khan, K., D. Cunningham, and I. Chau, Targeting Angiogenic Pathways in Colorectal Cancer: Complexities, Challenges and Future Directions. Curr Drug Targets, 2017. 18(1): p. 56-71.
47. Iacovelli, R., et al., Inhibition of the VEGF/VEGFR pathway improves survival in advanced kidney cancer: a systematic review and meta-analysis. Curr Drug Targets, 2015. 16(2): p. 164-70.
48. Piperdi, B., A. Merla, and R. Perez-Soler, Targeting angiogenesis in squamous non-small cell lung cancer. Drugs, 2014. 74(4): p. 403-13.
49. von Baumgarten, L., et al., Bevacizumab has differential and dose-dependent effects on glioma blood vessels and tumor cells. Clin Cancer Res, 2011. 17(19): p. 6192-205.
50. Yuan, F., et al., Time-dependent vascular regression and permeability changes in established human tumor xenografts induced by an anti-vascular endothelial growth factor/vascular permeability factor antibody. Proc Natl Acad Sci USA, 1996. 93(25): p. 14765-70.
51. Aalders, K. C., et al., Anti-angiogenic treatment in breast cancer: Facts, successes, failures and future perspectives. Cancer Treat Rev, 2017. 53: p. 98-110.
52. Kerbel, R. S., Reappraising antiangiogenic therapy for breast cancer. Breast, 2011. 20 Suppl 3: p. S56-60.
53. Kerbel, R. S., Issues regarding improving the impact of antiangiogenic drugs for the treatment of breast cancer. Breast, 2009. 18 Suppl 3: p. S41-7.
54. Lu, K. V. and G. Bergers, Mechanisms of evasive resistance to anti-VEGF therapy in glioblastoma. CNS Oncol, 2013. 2(1): p. 49-65.
55. Chen, J., et al., Severe Cardiotoxicity in a Patient with Colorectal Cancer Treated with Bevacizumab. Anticancer Res, 2017. 37(8): p. 4557-4561.
56. Economopoulou, P., et al., Cancer therapy and cardiovascular risk. focus on bevacizumab. Cancer Manag Res, 2015. 7: p. 133-43.
57. des Guetz, G., et al., Cardiovascular toxicity of anti-angiogenic drugs. Target Oncol, 2011. 6(4): p. 197-202.
58. Advani, R. H., et al., Cardiac toxicity associated with bevacizumab (Avastin) in combination with CHOP chemotherapy for peripheral T cell lymphoma in ECOG 2404 trial. Leuk Lymphoma, 2012. 53(4): p. 718-20.
59. Hawkes, E. A., et al., Cardiotoxicity in patients treated with bevacizumab is potentially reversible. J Clin Oncol, 2011. 29(18): p. e560-2.
60. Yardley, D. A., Integrating bevacizumab into the treatment of patients with early-stage breast cancer: focus on cardiac safety. Clin Breast Cancer, 2010. 10(2): p. 119-29.
61. Drimal, J., et al., Cardiovascular toxicity of the first line cancer chemotherapeutic agents: doxorubicin, cyclophosphamide, streptozotocin and bevacizumab. Neuro Endocrinol Lett, 2006. 27 Suppl 2: p. 176-9.
62. Rafii, S., et al., Angiogenic factors reconstitute hematopoiesis by recruiting stem cells from bone marrow microenvironment. Ann N Y Acad Sci, 2003. 996: p. 49-60.
63. Hattori, K., et al., Placental growth factor reconstitutes hematopoiesis by recruiting VEGFR1(+) stem cells from bone-marrow microenvironment. Nat Med, 2002. 8(8): p. 841-9.
64. DiNitto, J. P. and J. C. Wu, Molecular mechanisms of drug resistance in tyrosine kinases cAbl and cKit. Crit Rev Biochem Mol Biol, 2011. 46(4): p. 295-309.
65. Catalona, W. J., et al., Identification of complement-receptor lymphocytes (B cells) in lymph nodes and tumor infiltrates. J Urol, 1975. 114(6): p. 915-21.
66. Wolf, G. T., et al., Lymphocyte subpopulations infiltrating squamous carcinomas of the head and neck. correlations with extent of tumor and prognosis. Otolaryngol Head Neck Surg, 1986. 95(2): p. 142-52.
67. Holmes, E. C., Immunology of tumor infiltrating lymphocytes. Ann Surg, 1985. 201(2): p. 158-63.
68. Lipponen, P. K., et al., Tumour infiltrating lymphocytes as an independent prognostic factor in transitional cell bladder cancer. Eur J Cancer, 1992. 29A(1): p. 69-75.
69. Di Giorgio, A., et al., The influence of tumor lymphocytic infiltration on long term survival of surgically treated colorectal cancer patients. Int Surg, 1992. 77(4): p. 256-60.
70. Kawata, A., et al., Tumor-infiltrating lymphocytes and prognosis of hepatocellular carcinoma. Jpn J Clin Oncol, 1992. 22(4): p. 256-63.
71. Furihata, M., et al., Prognostic significance of simultaneous infiltration of HLA-DR-positive dendritic cells and tumor infiltrating lymphocytes into human esophageal carcinoma. Tohoku J Exp Med, 1993. 169(3): p. 187-95.
72. Sarioglu, T., et al., The effect of lymphocytic infiltration on clinical survival in cancer of the tongue. Eur Arch Otorhinolaryngol, 1994. 251(6): p. 366-9.
73. Slootweg, P. J., et al., Lymphocytes at tumor margins in patients with head and neck cancer. Relationship with tumor size, human lymphocyte antigen molecules, and metastasis. Int J Oral Maxillofac Surg, 1994. 23(5): p. 286-9.
74. Mihm, M. C., Jr., C. G. Clemente, and N. Cascinelli, Tumor infiltrating lymphocytes in lymph node melanoma metastases: a histopathologic prognostic indicator and an expression of local immune response. Lab Invest, 1996. 74(1): p. 43-7.
75. Clemente, C. G., et al., Prognostic value of tumor infiltrating lymphocytes in the vertical growth phase of primary cutaneous melanoma. Cancer, 1996. 77(7): p. 1303-10.
76. Hakansson, A., et al., Tumour-infiltrating lymphocytes in metastatic malignant melanoma and response to interferon alpha treatment. Br J Cancer, 1996. 74(5): p. 670-6.
77. Eerola, A. K., Y. Soini, and P. Paakko, Tumour infiltrating lymphocytes in relation to tumour angiogenesis, apoptosis and prognosis in patients with large cell lung carcinoma. Lung Cancer, 1999. 26(2): p. 73-83.
78. Prince, M. E. P., et al., Evaluation of the immunogenicity of ALDH(high) human head and neck squamous cell carcinoma cancer stem cells in vitro. Oral Oncol, 2016. 59: p. 30-42.
79. Huang, Z., et al., Correlation of cancer stem cell markers and immune cell markers in resected non-small cell lung cancer. J Cancer, 2017. 8(16): p. 3190-3197.
80. Codd, A. S., et al., Cancer stem cells as targets for immunotherapy. Immunology, 2017.
81. Rapp, C., et al., Identification of T cell target antigens in glioblastoma stem-like cells using an integrated proteomics-based approach in patient specimens. Acta Neuropathol, 2017. 134(2): p. 297-316.
82. Maccalli, C., G. Parmiani, and S. Ferrone, Immunomodulating and Immunoresistance Properties of Cancer-Initiating Cells: Implications for the Clinical Success of Immunotherapy. Immunol Invest, 2017. 46(3): p. 221-238.
83. Meng, M. B., et al., Enhanced radioresponse with a novel recombinant human endostatin protein via tumor vasculature remodeling: experimental and clinical evidence. Radiother Oncol, 2013. 106(1): p. 130-7.
84. Czarnota, G. J., et al., Tumor radiation response enhancement by acoustical stimulation of the vasculature. Proc Natl Acad Sci USA, 2012. 109(30): p. E2033-41.
85. Peng, F., et al., Recombinant human endostatin normalizes tumor vasculature and enhances radiation response in xenografted human nasopharyngeal carcinoma models. PLoS One, 2012. 7(4): p. e34646.
86. Zawaski, J. A., et al., Effects of irradiation on brain vasculature using an in situ tumor model. Int J Radiat Oncol Biol Phys, 2012. 82(3): p. 1075-82.
87. Truman, J. P., et al., Endothelial membrane remodeling is obligate for anti-angiogenic radiosensitization during tumor radiosurgery. PLoS One, 2010. 5(9).
88. Brose, K., et al., Slit proteins bind Robo receptors and have an evolutionarily conserved role in repulsive axon guidance. Cell, 1999. 96(6): p. 795-806.
89. Yadav, S. S. and G. Narayan, Role of ROBO4 signalling in developmental and pathological angiogenesis. Biomed Res Int, 2014. 2014: p. 683025.
90. Zhuang, X., et al., Robo4 vaccines induce antibodies that retard tumor growth. Angiogenesis, 2014.
91. Paprocka, M., et al., CD133 positive progenitor endothelial cell lines from human cord blood. Cytometry A, 2011. 79(8): p. 594-602.
92. Chen, C., et al., Adult endothelial progenitor cells retain hematopoiesis potential. Transplant Proc, 2010. 42(9): p. 3745-9.
93. Smadja, D. M., et al., Increased VEGFR2 expression during human late endothelial progenitor cells expansion enhances in vitro angiogenesis with up-regulation of integrin alpha(6). J Cell Mol Med, 2007. 11(5): p. 1149-61.
94. Ziegler, B. L., et al., KDR receptor: a key marker defining hematopoietic stem cells. Science, 1999. 285(5433): p. 1553-8.
95. Yan, H. X., et al., Active immunotherapy for mouse breast cancer with irradiated whole-cell vaccine expressing VEGFR2. Oncol Rep, 2013. 29(4): p. 1510-6.
96. Feng, K., et al., Anti-angiogenesis effect on glioma of attenuated Salmonella typhimurium vaccine strain with flk-1 gene. J Huazhong Univ Sci Technolog Med Sci, 2004. 24(4): p. 389-91.
97. Wada, S., et al., Rationale for antiangiogenic cancer therapy with vaccination using epitope peptides derived from human vascular endothelial growth factor receptor 2. Cancer Res, 2005. 65(11): p. 4939-46.
98. Yan, J., et al., A promising new approach of VEGFR2-based DNA vaccine for tumor immunotherapy. Immunol Lett, 2009. 126(1-2): p. 60-6.
99. Ren, S., et al., Inhibition of tumor angiogenesis in lung cancer by T4 phage surface displaying mVEGFR2 vaccine. Vaccine, 2011. 29(34): p. 5802-11.
100. Wei, Y., et al., Enhancement of DNA vaccine efficacy by targeting the xenogeneic human chorionic gonadotropin, survivin and vascular endothelial growth factor receptor 2 combined tumor antigen to the major histocompatibility complex class II pathway. J Gene Med, 2012. 14(5): p. 353-62.
101. Suzuki, H., et al., Multiple therapeutic peptide vaccines consisting of combined novel cancer testis antigens and anti-angiogenic peptides for patients with non-small cell lung cancer. J Transl Med, 2013. 11: p. 97.
102. Facciponte, J. G., et al., Tumor endothelial marker 1-specific DNA vaccination targets tumor vasculature. J Clin Invest, 2014. 124(4): p. 1497-511.
103. Lladser, A., et al., Intradermal DNA electroporation induces survivin-specific CTLs, suppresses angiogenesis and confers protection against mouse melanoma. Cancer Immunol Immunother, 2010. 59(1): p. 81-92.
104. Xiang, R., et al., Oral DNA vaccines target the tumor vasculature and microenvironment and suppress tumor growth and metastasis. Immunol Rev, 2008. 222: p. 117-28.
105. Xiang, R., et al., A DNA vaccine targeting survivin combines apoptosis with suppression of angiogenesis in lung tumor eradication. Cancer Res, 2005. 65(2): p. 553-61.
106. Valdez, Y., M. Mala, and E. M. Conway, CD248: reviewing its role in health and disease. Curr Drug Targets, 2012. 13(3): p. 432-9.
107. Plum, S. M., et al., Generation of a specific immunological response to FGF-2 does not affect wound healing or reproduction. Immunopharmacol Immunotoxicol, 2004. 26(1): p. 29-41.
108. Wei, Y. Q., et al., Immunogene therapy of tumors with vaccine based on Xenopus homologous vascular endothelial growth factor as a model antigen. Proc Natl Acad Sci USA, 2001. 98(20): p. 11545-50.
109. Liu, J. Y., et al., Immunotherapy of tumors with vaccine based on quail homologous vascular endothelial growth factor receptor-2. Blood, 2003. 102(5): p. 1815-23.
110. Su, J. M., et al., Active immunogene therapy of cancer with vaccine on the basis of chicken homologous matrix metalloproteinase-2. Cancer Res, 2003. 63(3): p. 600-7.
111. Tan, G. H., et al., Active immunotherapy of tumors with a recombinant xenogeneic endoglin as a model antigen. Eur J Immunol, 2004. 34(7): p. 2012-21.
112. Jiao, J. G., et al., A plasmid DNA vaccine encoding the extracellular domain of porcine endoglin induces anti-tumour immune response against self-endoglin-related angiogenesis in two liver cancer models. Dig Liver Dis, 2006. 38(8): p. 578-87.

1. A hybrid cell comprising of: a) a dendritic or dendritic like cell and b) an endothelial cell generated in a manner to resemble tumor endothelium.

2. The hybrid cell of claim 1, wherein said hybrid cells is generated by fusion of a dendritic or dendritic like cell and an endothelial cell generated in a manner to resemble tumor endothelium.

3. The hybrid cell of claim 3, wherein said fusion is created by placement of both cell types in physical proximity while treating both cells with an agent capable of causing fusion of plasma membrane.

4. The hybrid cell of claim 3, wherein said fusion agent is one or more agents selected from a group comprising of: a) polyethylene glycol; b) ultrasound waves; c) radio waves; and d) phosphatidylcholine.

5. The hybrid cell of claim 1, wherein said dendritic cell is generated from a group of cells comprising of: a) a stem cell; b) a pluripotent stem cell; c) an inducible pluripotent stem cell; d) a parthenogenic stem cell; e) a somatic cell nuclear transfer derived stem cell; f) a pluripotent stem cell is generated by cytoplasmic transfer from an immature cell to a mature cell; and g) embryoid bodies from said pluripotent stem cells.

6. The hybrid cell of claim 5, wherein said embryoid bodies are dissociated and cells are cultured in cytokines capable of expanding dendritic cell progenitors.

7. The hybrid cell of claim 5, wherein said dendritic cell progenitors are cultured in GM-CSF.

8. The hybrid cell of claim 5, wherein said dendritic cell progenitors are cultured in flt-3 ligand.

9. The hybrid cell of claim 5, wherein said dendritic cell progenitors are cultured in IL-4.

10. The hybrid cell of claim 1, wherein said endothelial cells are derived from a pluripotent stem cell.

11. The hybrid cell of claim 10, wherein said pluripotent stem cell is an inducible pluripotent stem cell.

12. The hybrid cell of claim 10, wherein said pluripotent stem cell is a parthenogenic stem cell.

13. The hybrid cell of claim 10, wherein said pluripotent stem cell is a somatic cell nuclear transfer derived stem cell.

14. The hybrid cell of claim 10, wherein said pluripotent stem cell is generated by cytoplasmic transfer from an immature cell to a mature cell.

15. The hybrid cell of claim 1, wherein said hybrid cell is utilized to induce an immune response against tumor endothelial cells.

16. The hybrid cell of claim 10, wherein said endothelial cells are generated by culture of endothelial progenitor cells in a media replicating the tumor microenvironment.

17. The hybrid cell of claim 10, wherein said endothelial cells are generated by culture of endothelial progenitor cells in a media replicating the tumor microenvironment.

18. The hybrid cell of claim 17, wherein said media contains one or more agents selected from a group comprising of: a) prostaglandin E2; b) TGF-beta; c) IL-10; d) VEGF; e) PDGF-BB; f) EGF; g) FGF-1 and h) FGF-2.

19. The hybrid cells of claim 1, wherein antigen presenting activity of said cells is augmented as compared to baseline conditions by treatment with a toll like receptor agonist.

20. The hybrid cells of claim 19, wherein said toll like receptor agonist is HMGB-1 or a peptide derived thereof.
